(19) United States
(12) Patent Application Publication
(10) Pub. No.: US 2019/0087540 A1
(43) Pub. Date: Mar. 21, 2019

(54) SYSTEM AND METHOD FOR ANALYZING GENOTYPE USING GENETIC VARIATION INFORMATION ON INDIVIDUAL'S GENOME

(71) Applicant: SYNTEKABIO CO., LTD., Daejeon (KR)

(72) Inventor: Jongsun JUNG, Daejeon (KR)

(73) Assignee: SYNTEKABIO CO., LTD., Daejeon (KR)

(21) Appl. No.: 16/065,982

(22) PCT Filed: Dec. 28, 2016
(86) PCT No.: PCT/KR2016/015389

§ 371 Date: Nov. 30, 2018

(30) Foreign Application Priority Data
Dec. 28, 2015 (KR) 10-2015-0187556

(51) Int. Cl. G06F 19/20; G16H 10/40; G16H 50/70; G16H 50/30; G16H 50/20

(52) U.S. Cl. CPC G06F 19/28; G06F 19/18; G06F 19/24; G16H 50/20; G16H 10/40; G16H 50/70; G16H 50/30; G06F 19/20

(57) ABSTRACT

Disclosed is a system for genotype analysis using genetic variation information on a personal genome. The system includes an analysis data input unit configured to receive analysis data including personal genomic information; a search control unit configured to produce analysis results including a genotype of each gene or genotype versus phenotype by comparing genetic information stored in a database with the analysis data and to generate a result report based on the analysis results; and a storage unit comprising a haplotype DB that stores genotype information on genes of a control group to compare with the analysis data. The search control unit includes a HaploScan engine configured to determine the genotype of the analysis date by comparing the analysis data with the haplotype DB.

### CROSS-REFERENCE TO PRIOR APPLICATIONS

This application is a National Stage Patent Application of PCT International Patent Application No. PCT/KR2016/015389 filed on Dec. 28, 2016 under 35 U.S.C. §371, which claims priority to Korean Patent Application No. 10-2015-0187556 filed on Dec. 28, 2015, which are all hereby incorporated by reference in their entirety.

### BACKGROUND

The present invention relates to a method and a system of analyzing and providing genotype information from a personal genome by comparing input personal genome information with a plurality of genome DBs constructed by genome projects.

The current IT market trends are changing in the order of Google, Facebook, Amazon, cloud computing and Ubiquitous, and at the same time, biomedical, bioinformatics and genomics are also changing according to new trends in the order of bio-Google, system bio, personalized medicine and precision medicine. Particularly, in the Post-Human Genome Project era, the next generation sequencing technology has been developed rapidly and efforts have been actively made to realize individualized/personalized medicine.

Currently, the next generation sequencing technology is known to take about one week to sequence (decode) and analyze the whole genome of a person (x30). In addition, it was reported that about 100,000 next-generation sequencers were supplied worldwide, and it was that a significant amount of money has been invested in major companies which have developed the third-generation sequencer (Ion Torrent: 2.5 generation; Pacific BioScience: third generation).

In addition, this field is the fastest advancing and developing field among all businesses in the world. As this trend progresses, the cost for sequencing and analyzing the whole genome of a person is expected to decrease to less than approximately $1,000 within the next two to three years. The most useful and immediately practicable technologies based on the above next generation technologies are clinical genomics, pharmaco-genomics and translational medicine. In addition, such clinical genomics has recently been applied to medical genomics, and such medical genomics, along with patient stratification technologies, have created a new discipline and new language called Precision Medicine mentioned by U.S. President Obama.

As described above, information on genetic variation is increasing every year, and the area of analysis accuracy will be continuously expanded by expansion of verified data according to the present invention.

Meanwhile, the applicant has continued to develop technology in order to improve the technical requirements of the above-mentioned genetic analysis field.

As a result of these efforts, the applicant has developed methods for precision medicine, clinical information, proteome and genome information related to bio-big data, and construction of analysis systems for increasing the analysis speed thereof. In particular, the applicant developed a GPU (graphic process unit)-based analysis system for analysis speed (Korean Patent No. 10-0996443), and developed information searching methods based on characteristic files of an RVR (records virtual rack) analysis tool which is a technique for increasing data comparison speed (Korean Patent Nos. 10-0880531, 10-1035959 and 10-1117603).

In addition, the applicant applied RVR and GPU (graphic process unit) to proteomes (Korean Patent No. 10-1400717), and developed allele depth-based ADISCAN analysis tools for efficiently determining variant calling and the level of rare variation between a control and an individual genome (Korean Patent No. 10-1460520, 10-1542529 and 10-2014-0020738).

In addition, the applicant developed methods for construction of an integrated genome DB for efficiently managing genome information, identification of mutations for disease causes, and genotype calculation for patient stratification (Korean Patent Nos. 10-2015-0187554, 10-2015-0187556 and 10-2015-0187559), and a method for computing human haplotyping from genome information (Korean Patent Application No. 10-2016-0096996).

In addition, using middleware specialized for storage of big data such as integrated genetic DB, MAHA supercomputing systems were developed which enables thousands of genomic bulk data to be analyzed simultaneously in a parallel distributed environment developed by the Electronics and Telecommunications Research Institute (ETRI) (Korean Patent Nos. 10-1460520, 10-1010219, 10-0956637, 10-0936238, 10-2013-0005685, 10-2012-0146892 and 10-2013-0004519).

Using the MAHA system provided from the Electronics and Telecommunications Research Institute, the applicant has developed the first domestic supercomputing system, which has an optimized environment utilizing bio big data for clinical applications and is integrated with an integrated genome analysis system for precision medicine implementation.

In particular, although MAHA-Fs (a storage system for ultrahigh speed I/O for bulk data such as genome) was tailored to a common cloud computing environment, the applicant has developed MAHA-FsDx, which can be used for diagnosis in a clinical environment, that is, a hospital, by clearly defining reproducibility, precision and system limitations. In addition, the following prior tool-related patents and patent applications (001) to (019) owned by the applicant summarize the technical elements for a personal genome map(PMAP)-based personalized medical analysis platform.

### LIST OF PRIOR ART PATENT DOCUMENTS

(Patent document 1) (001) Korean Patent No. 10-0880531;

(Patent document 2) (002) Korean Patent No. 10-0996443;

(Patent document 3) (003) Korean Patent No. 10-1035959;

(Patent document 4) (004) Korean Patent No. 10-1117603;

(Patent document 5) (005) Korean Patent No. 10-1400717;

(Patent document 6) (006) Korean Patent No. 10-1460520;

(Patent document 7) (007) Korean Patent No. 10-1542529;

(Patent document 8) (008) Korean Patent Application No. 10-2015-0187554;

(Patent document 9) (009) Korean Patent Application No. 10-2015-0187556;

(Paten document 10) (010) Korean Patent Application No. 10-2015-0187559;

(Patent document 11) (011) Korean Patent Application No. 10-2016-0096996;

(Patent document 12) (012) Korean Patent No. 10-0834574;

(Patent document 13) (013) Korean Patent No. 10-1010219;

(Patent document 14) (014) Korean Patent No. 10-0956637;

(Patent document 15) (015) Korean Patent No. 10-0936238;

(Patent document 16) (016) Korean Patent Application No. 10-2013-0005685;

(Patent document 17) (017) Korean Patent Application No. 10-2012-0146892;

(Patent document 18) (018) Korean Patent Application No. 10-2013-0004519;

(Patent document 19) (019) Korean Patent Application No. 10-2016-0172053.

### SUMMARY

The present invention has been made in order to improve requirements for realizing personal genomic personalized medicine based on the “personal genome map-based personalized medical analysis platform” as described above, and is intended to provide a genotyping platform utilizing a database schema capable of increasing the detection speed and efficiency of a standardized ID set based on personal genome analysis (haplotype IDs with various genotypes, personal profile) and hospital clinical information (a specific phenotype or various phenotypes).

The present invention is also intended to provide a system for generating a standardized ID set, which provides information about the genotype of detected genome (or personal profile) so as to be easily recognized by the user.

A system for computing the cause of disease and drug (or food) response calculates multiple regression analysis coefficients based on population genetic information and clinical information, and calculates a relationship index (pi, Π), which is the result of logistic regression, by use of personal genetic information and clinical information as variables. In this regard, the relationship index (pi, Π) is calculated by receiving a standardized ID set based on personal genome analysis (genotype marker ID) and hospital clinical information (a specific genotype or various genotypes) and using the values as input. In addition, when the relationship index (pi, Π) is in the range of 0.7 to 1, the specific genetic marker ID of the person becomes the direct (or indirect) cause of a given phenotype.

As shown in FIG. 1, the system for identifying the cause of disease and drug (or food) response according to the present invention generally comprises a personal genome analysis platform, an integrated genome DB, a unit for computing the cause of personal genome-based disease (drug) response, and an algorithm for computing the cause of disease (drug) response.

The personal genome analysis platform comprises \textcircled{1} to \textcircled{5} of FIG. 1. Regarding this, the standardized ID set system uses the term “genotype (trait) calculation”. Although scientists may have different opinions, the definition of (genotype) trait in this patent is determined by a standardized ID set and similar methods.

Namely, the standardized ID set refers to haplotyping-based LD block haplotype ID, Exon haplotype ID, gene marker haplotype ID, multiple gene marker haplotype ID, GWAS marker haplotype ID, BAV (bio active variant) marker ID of physiologically active single variations or sets in this patent, and ID in markers in a common independent (or individual) biomarker DB, and it includes GWAS markers, Clinvar markers, eQTL markers, proteome markers, STR markers, Fusion markers, and the like.

In addition, it includes diagnostic phenotype information such as electronic medical records (EMRs), electronic health records (EHRs) and personal health records (PHRs), etc., held by hospitals or medical examination centers.

In addition, it includes drug clinical phenotype information such as drug responders/non-responders of drug and health food (or food) clinical (IIT: investigator initiative clinical trial, SIT: sponsor initiative clinical trial, PMS: post-market survey).

In addition, the integrated genomic DB comprises of FIG. 1, and it refers to a database for calculating coefficient values using the integrated genomic DB and the standard phenotype disease information included in hospital medical systems. Here, different multiple coefficient values per phenotype are calculated, and if necessary, multiple coefficient values for multiple phenotypes may be calculated.

Furthermore, the unit for computing the cause of personal genome-based disease (drug) response comprises of FIG. 1, and functions to compute information on personal genome and hospital phenotypes.

Thus, as information on personal genome and hospital phenotypes is given, the relationship index (pi, Π) is obtained by the algorithm for calculating the cause of disease (drug) response.

The relationship index (pi, Π) is the result of multiple logistic regression. The relationship index (Π) is given as a probability score from 0 to 1. A relationship index close to 0.7-1 indicates that a probability of having a given phenotype is high, and a relationship index of 0-0.3 is opposite to a given phenotype. In addition, a relationship index of 0.4-0.6 indicates that the phenotype is in an intermediate stage.

In particular, haplotyping-based haplotypes include LD (linkage disequilibrium) block haplotypes, Exon haplotypes, gene marker haplotypes, multiple gene marker haplotypes, and GWAS (genome wide association study) marker haplotypes. For common points in haplotypes, haplotyping of specific units of human genes is performed, and among them, only important markers (e.g., GWAS markers) may be used, or the whole sequence (exon, gene, or LD block) may be used. The haplotype ID generated as described above may be named trait which is a generic term. In particular, haplotyping-based haplotypes may also be used as human standardized ID sets.

Meanwhile, the present invention provides a system for genotype analysis, comprising: an analysis date input unit configured to receive analysis data including personal genomic information; a search control unit configured to produce analysis results including the genotype of each gene or genotype versus phenotype by comparing genetic information stored in a database with the analysis data and to generate a result report based on the analysis results; and a storage unit comprising a haplotype DB that stores genotype information on a control gene to compare with the analysis data. The search control unit comprises a HaploScan engine configured to determine the genotype of the analysis date by comparing the analysis data with the haplotype DB. The haplotype DB comprises: a single-gene information database that stores genotype information on single genes; a multiple-gene information database that stores genotype information on multiple genes for each genotype. The single-gene information database comprises: a single-map haplo map that stores haplotype and trait frequencies for each race, classified (clustered) by proportion, for single genes of a control group; and single-gene haplo frequency information configured to store variation information on variations that classify the single-gene genotypes stored in the single-gene haplo map. The multiple-gene information database comprises: a multiple-gene haplo map that stores genotype-associated nucleotide variation distributions classified by race and proportion, for multiple genes of a control group for each phenotype; and multiple-gene haplo frequency information configured to store variation information on variations that classify genotypes for the phenotypes stored in the multiple-gene haplo map. The storage unit further comprises a clinical information DB that stores subject's environmental factor information to be considered together with genetic traits in order to produce the results of disease cause prediction based on clinical information. Here, the search control unit can produce the results of disease cause prediction by generating the relationship index (Π) for disease cause relationship through an arithmetic expression generated by multiple logistic regression.

In addition, the arithmetic expression for disease cause or drug response relationship is

$${π}_{x} = \frac{exp ( {β}_{0} + {β}_{1} {x}_{1} + {β}_{2} {x}_{2} + … + {β}_{n} {x}_{n} )}{1 + exp ( {β}_{0} + {β}_{1} {x}_{1} + {β}_{2} {x}_{2} + … + {β}_{n} {x}_{n} )} ,$$

and is calculated using genotypes or a variety of given ID generation systems, given in personal profiles (standardized ID set), through population genomes and their EMR (electronic medical record), EHR (electrical health record) and PHR (personal health record). In addition, coefficient variables β are generated using a given ID system. Furthermore, personal information generates personal profiles (standardized ID set) by using the personal genome and the hospital-based information on the person as standards, and the IDs provide variable χ to the arithmetic expression determined by multiple logistic regression.

Here, the result report may also comprise an index indicating the level of significance compared with the classified region (class) to which the genotype of the analysis date belongs.

Meanwhile, the present invention provides a method for genotype analysis, comprising: step (A) in which an analysis date input unit receives analysis data consisting of DNA sequencing; step (B) in which a HaploScan engine determines the genotype of a gene of the analysis data; step (C) in which the HaploScan engine acquires variation information on the gene of the analysis data; step (D) in which step (B) and step (C) are repeatedly performed on all genes included in the analysis data; and step (E) in which a search control unit produces the results of disease cause prediction by generating a disease cause relationship (Πx) through an arithmetic expression generated by logistic regression, wherein the determination of the genotype in step (B) comprises: a step of determining the genotype of interest among genotypes classified in a single-gene haplo map, for single genes of the analysis data; and a step of determining the genotype of interest among genotypes classified in a multiple-gene haplo map, for multiple genes included in the analysis data; the acquisition of the variation information in step (C) comprises: a step of comparing single-gene haplo frequency information on a specific locus gene of the analysis data with that on the same locus gene, thereby acquiring variation information on a specific locus gene of the analysis data; and comparing multiple-gene haplo frequency information on multiple genes of the analysis data with that on a specific phenotype, thereby acquiring variation information on the multiple genes of the analysis data; the single-gene haplo map stores haplotype and trait frequencies for each race, classified (clustered) by proportion, for single genes of a control group; the multiple-gene haplo frequency information stores variation information on variations that classify the single-gene genotypes stored in the single-gene haplo map; the multiple-gene haplo map stores multiple-gene variation distributions classified by proportion, for multiple genes of a control group for each phenotype; the multiple-gene haplo frequency information is variation information on variations that classify genotypes for the phenotypes; and the arithmetic expression for disease cause or drug (or food) response is

$${π}_{x} = \frac{exp ( {β}_{0} + {β}_{1} {x}_{1} + {β}_{2} {x}_{2} + … + {β}_{n} {x}_{n} )}{1 + exp ( {β}_{0} + {β}_{1} {x}_{1} + {β}_{2} {x}_{2} + … + {β}_{n} {x}_{n} )} ,$$

and is calculated using genotypes or a variety of given ID generation systems, given in personal profiles (standardized ID set), through population genomes and their EMR (electronic medical record), EHR (electrical health record) and PHR (personal health record). Coefficient variables β are generated using a given ID system. Furthermore, personal information generates personal profiles (standardized ID set) by using the personal genome and the hospital-based information on the person as standards, and the IDs provide variable χ to the arithmetic expression determined by multiple logistic regression.

The method according to the present invention may further comprise step (F) in which the search control unit generates a result report through the obtained result.

Furthermore, the result report may further comprise an index indicating the level of significance compared with the classified region (class) to which the genotype of the analysis data belongs.

The system of identifying disease causes using genetic information on genetic variation of a personal genome according to the present invention as described above has the effect of rapidly and efficiently performing the determination of the genotype of the personal genome (or personal profiles, standardized ID sets) by effectively comparing genetic variation information stored in a control database with that on the personal genome to be analyzed.

### BRIEF DESCRIPTION OF THE DRAWINGS

FIG. 1 is a conceptual view showing the conceptual configuration of a system for computing the cause of disease and drug response according to the present invention.

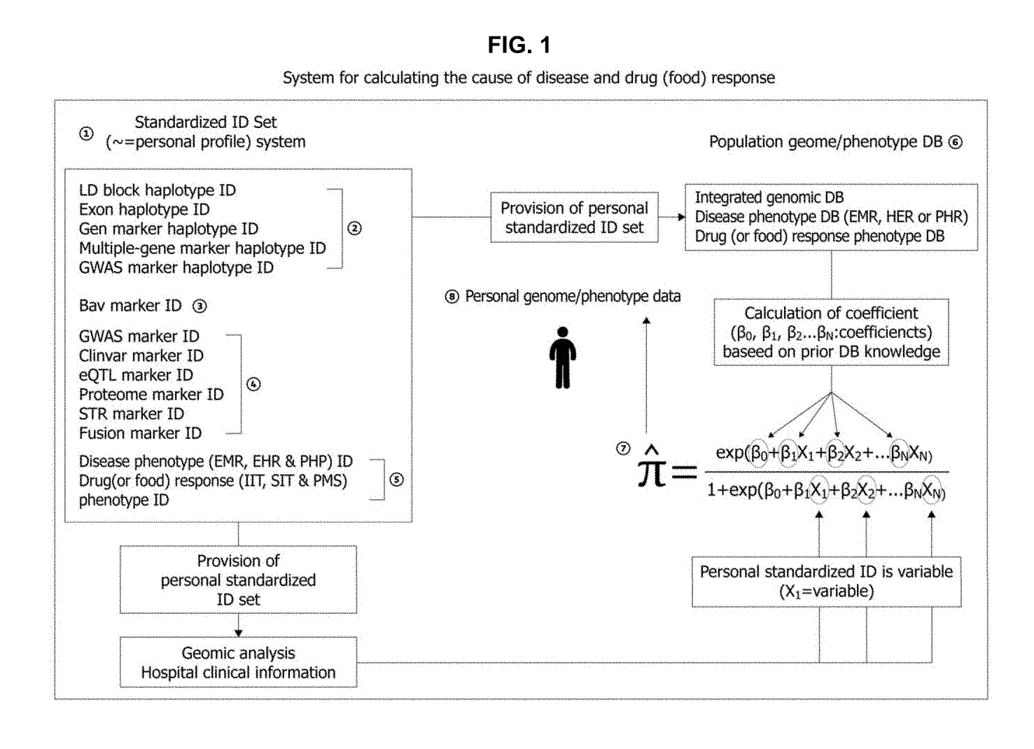

FIG. 2 illustrates the configuration of a genetic analysis service utilizing the present invention.

FIG. 3 is a block diagram showing a genotype analysis system according to a specific embodiment of the present invention.

FIG. 4 illustrates major databases constituting a system for identifying disease causes according to the present invention.

FIG. 5 is a conceptual view showing an example of the configuration of a haplo map according to a specific embodiment of the present invention.

FIG. 6 shows an example of the configuration of a haplotype DB according to a specific embodiment of the present invention.

FIG. 7 is a flow chart showing a genotype analysis method according to a specific embodiment of the present invention.

FIG. 8 illustrates an example for generating a haplotype DB according to a specific embodiment of the present invention.

FIG. 9 illustrates an example of genotyping results produced according to a specific embodiment of the present invention.

FIG. 10 illustrates an example of a Manhattan plot of a result report produced according to a specific embodiment of the present invention.

FIG. 11 illustrates an example of a radar mutation significance chart of a result report produced according to a specific embodiment of the present invention.

FIG. 12 illustrates another example of a radar mutation significance chart of a result report produced according to a specific embodiment of the present invention.

FIG. 13 is a conceptual view showing a system for computing the cause of disease and drug (food) response based on clinical information according to a specific embodiment of the present invention.

### DETAILED DESCRIPTION

The present invention provides a system for genotype analysis, comprising: an analysis date input unit configured to receive analysis data including personal genomic information; a search control unit configured to produce analysis results including the genotype of each gene or genotype versus phenotype by comparing genetic information stored in a database with the analysis data and to generate a result report based on the analysis results; and a storage unit comprising a haplotype DB that stores genotype information on genes of a control group in order to compare with the analysis data.

In the system, the search control unit comprises a HaploScan engine configured to determine the genotype of the analysis date by comparing the analysis data with the haplotype DB, and the haplotype DB preferably comprises: a single-gene information database configured to store genotype information on single genes; and a multiple-gene information database configured to store genotype information on multiple genes for each genotype.

Furthermore, the single-gene information database comprises: a single-map haplo map that stores haplotype and trait frequencies for each race, classified (clustered) by proportion, for single genes of the control group; and single-gene haplo frequency information configured to store variation information on variations that classify the single-gene genotypes stored in the single-gene haplo map.

In addition, the multiple-gene information database comprises: a multiple-gene haplo map that stores genotype-associated nucleotide variation distributions classified by race and proportion for multiple genes of the control group for each phenotype; and multiple-gene haplo frequency information configured to store variation information on variations that classify genotypes for the phenotypes stored in the multiple-gene haplo map.

In addition, the storage unit preferably further comprises a clinical information DB configured to store subject's environmental factor information to be considered together with genetic traits in order to produce the results of disease cause prediction based on clinical information.

Hereinafter, a system and a method for genotype analysis using genetic variation information on a personal genome according to the present invention will be described in detail with reference to the accompanying drawings.

First, the construction of a genetic analysis service utilizing a system for identifying disease causes according to the present invention will be described briefly.

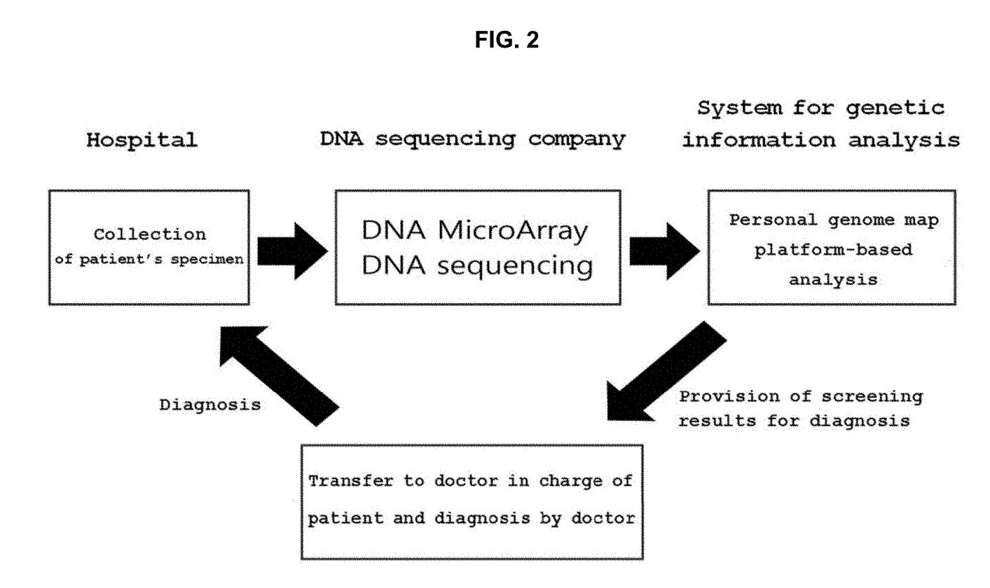

As shown in FIG. 2, in the genetic analysis service, a sample such as blood is collected from a personal gene collection agent such as a hospital, and the sample is transferred to a DNA sequencing company for diagnosis.

Then, the DNA sequencing company constructs a DNA custom chip from the collected sample or performs DNA sequencing (NGS, next generation sequencing). Of course, since DNA sequences can be generated by various methods as a result of recent technological developments, the DNA sequencing can be performed by various methods according to the technology level of the DNA sequencing company.

The DNA sequence generated as described above is analyzed by the system for genetic information analysis as described in the present invention, thereby analyzing genetic information included in the personal genome.

At this time, the system for genetic information analysis according to the present invention analyzes genetic information based on a personal genome map platform.

The analyzed information is transmitted to a diagnostic institution such as a hospital or a consumer.

Of course, as the DNA analysis data are provided from the DNA sequencing company, the system for identifying disease causes according to the present invention forms a highly integrated index file from the data and analyzes the genomic nucleotide sequence which is big data.

This will be described again below with reference to FIG.

Namely, the present invention is a system for genotype analysis which analyzes genetic information included in a personal genome from DNA sequencing information. Hereinafter, the system for genotype analysis according to the present invention will be described in detail.

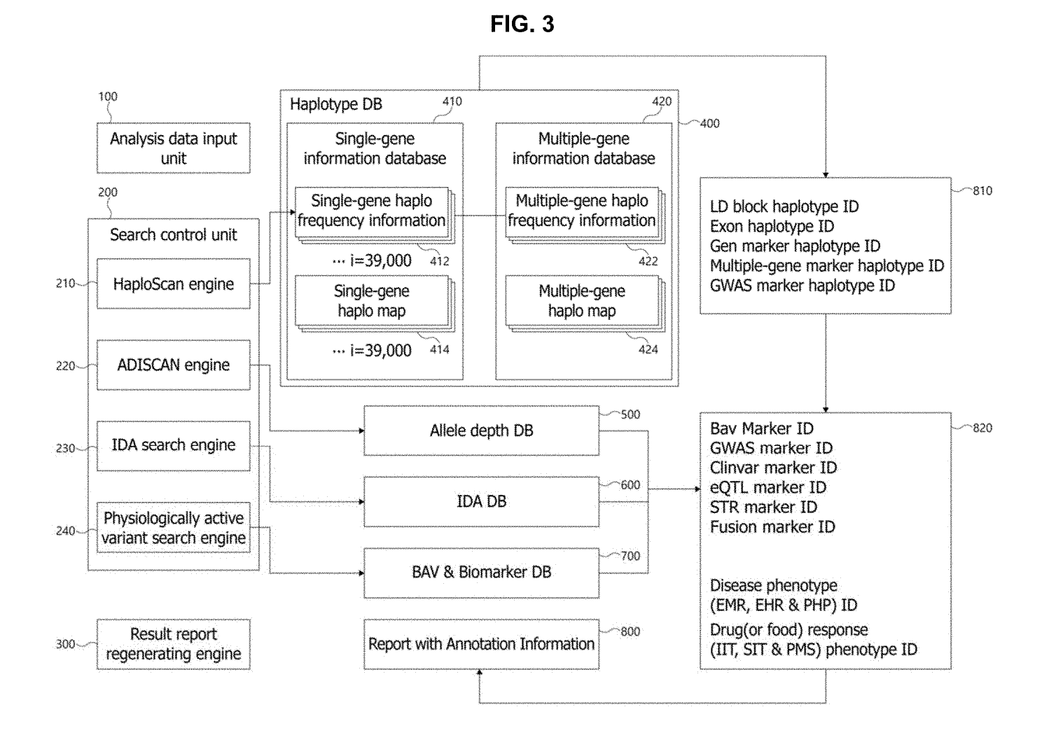

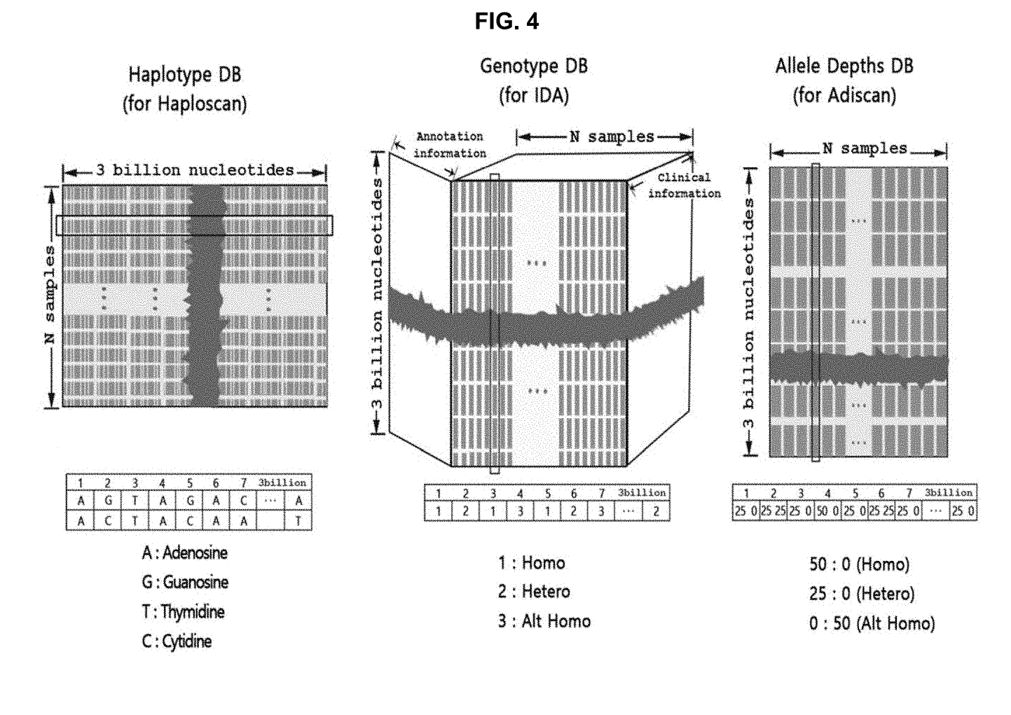

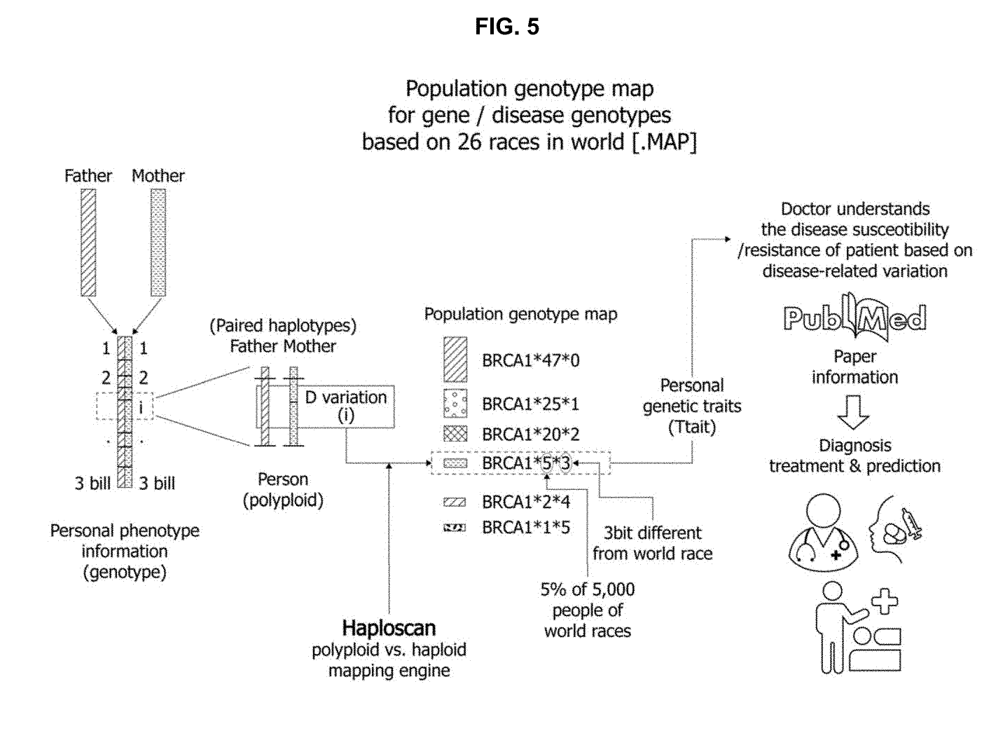

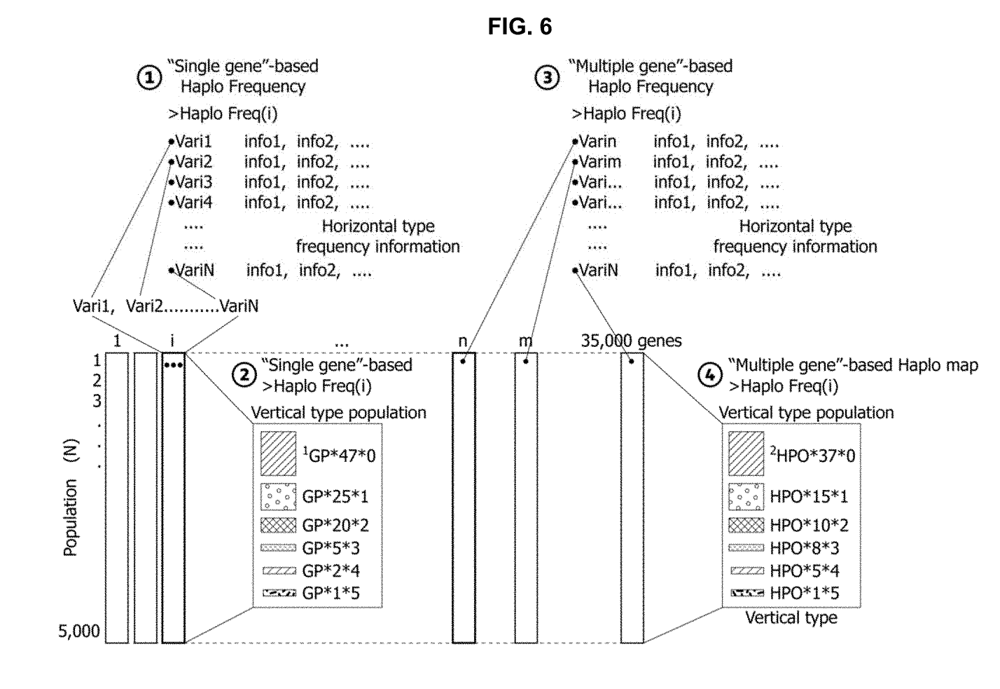

FIG. 3 is a block diagram showing the major components of a system for genotype analysis according to a specific embodiment of the present invention; FIG. 4 illustrates the configuration of major databases included in a system for identifying disease causes according to the present invention; FIG. 5 is a conceptual view showing an example of the construction of a haplo map according to a specific embodiment of the present invention; and FIG. 6 is a configurational view showing an example of the configuration of a haplotype DB according to a specific embodiment.

As shown in FIG. 3, a system for genotype analysis according to the present invention comprises a analysis data input unit 100, a search control unit 200, a result report provision unit 300, a haplotype DB 400, and an information DB 800, and may further comprise an allele depth DB 500, an IDA DB 600, a BAV/biomarker DB 700, a haplo ID generating unit 810, and a marker ID generating unit 820.

The analysis data input unit 100, a portion configured to receive personal genomic information, receives DNA sequencing data.

The search control unit 200 is configured to detect the genotype of each gene and genotype versus phenotype from the input sequencing data. To this end, the search control unit 200 comprises a HaploScan engine 210.

In addition, the search control unit 200 may further comprise an ADISCAN engine 220, an IDA search engine 230 and a physiologically active variant search engine 240 in order to detect rare variants, disease variants and physiologically active variants.

The HaploScan engine 210 is configured to determine the genotype by comparing the analysis data (input DNA sequencing data) with haplo maps 414 and 424 stored in a haplotype DB 400 to be described below.

The structure of the haplotype DB 400 and the method of search by the HaploScan engine 210 will be described again in detail below.

In addition, the ADISCAN engine 220 is configured to determine rarity compared to a population control group by comparing each base included in the input analysis data with the allele depth DB 500 by the ADISCAN method.

Furthermore, the IDA search engine 230 is configured to detect already known gene-related disease variants, and detects disease variants by comparing the analysis data with the IDA DB 600 that stores known disease variants.

In addition, the physiologically active variant search engine 240 is configured to detect protein metabolism-related genetic variants, and determines genetic variation for amino acids which are involved in protein-drug binding, protein-DNA binding and protein-protein binding.

At this time, the physiologically active variant search engine 240 compares the analysis data with the BAV/biomarker DB 700, thereby determining variation of nucleotides of the analysis data, which correspond to protein binding-related amino acids stored in the BAV/biomarker DB 700.

Meanwhile, the search control unit 200 generates a result report by use of a Manhattan plot and a radar variance significance chart so that the genotype determined by the HaploScan engine 210 can be visibly easily seen by a diagnoser (or user).

The generated result report is provided to the user through the result report provision unit 300.

Namely, the search control unit 200 generates haplotype IDs, including LD block haplotype ID, Exon haplotype ID, gene marker haplotype ID, multiple-gene marker haplotype ID and GWAS marker haplotype ID, through the haplo ID generation unit 400, based on the haplotype DB 400, and generates marker IDs, including Bav marker ID, GWAS marker ID, Clinvar maker ID, eQTL marker ID, proteome marker ID, STR marker ID, Fusion marker ID and the like, through the marker ID generating unit 820.

In this regard, a collection of the resulting IDs (which can be expressed as barcodes) is referred to as ‘standardized ID set (personal profile)’.

In addition, the final results are provided together with information (relationship index Π) on various disease/drug response causes and susceptibility results for IDs.

Hereinafter, the structure of the databases of the system for genotype analysis according to the present invention will be described.

The system for genotype analysis according to the present invention generally comprises a haplotype DB 400, an allele depth DB 500, an IDA DB 600, a BAV/biomarker DB 700, and an information DB 800.

Namely, as shown in FIG. 4, the integrated genome DB according to the present invention comprises a haplotype DB, an allele depth DB and an IDA DB. The haplotype DB is a DB generated by formatting all nucleotides in the IUPAC format, and the genotype & phenotype DB is a DB that comprises genotype and phenotype information and is configured to make it possible to detect disease relationship information, various correlations and QC. The allele depth DB is a DB for variant rarity and verification calculation.

As shown in FIG. 4, the haplotype DB 400 is a DB that summarizes the genotypes of genes of a control group in order to determine a genotype from the personal genomic information to be analyzed. As shown in FIG. 3, the haplotype DB 400 comprises a single-gene information database 410 and a multiple-gene information database 420.

Before describing the configuration of the haplotype DB, the fundamental configuration of the haplo map will now be described. As shown in FIG. 5, the Haplo map indicates classes divided by the genotypic proportion of each gene in the whole haploid genome of each of 5000 people of the world races, and includes the proportion of each genotype in the control group and difference values.

Thus, as shown in FIG. 5, using the personal genome (polyploid) of the analysis data, the prescriber (physician) can grasp by comparing paired haplotypes with the haplo map, and can provide academic information for diagnosis and treatment (prediction) of the subject (patient).

Meanwhile, as shown in FIG. 6, the haplotype DB (400) comprises a single-gene information database 410 and a multiple-gene information database 420. The single-gene information database 410 is a database that stores genotypes for single genes, and comprises a single-gene haplo map 414 and single-gene haplo frequency information 412.

Meanwhile, the single-gene haplo map 414 stores variation distributions classified (clustered) by proportion, for the same genes of the entire control group, and summarizes the results of calculating the haplotypes of 26 world races by use of each gene and calculating the frequency of a specific trait and the frequency of each sub-race.

In addition, the single-gene haplo frequency information 412 stores information on each variation. In this regard, the single-gene haplo frequency information 412 may be data that stores variation information, and information stored in the information DB 800 to be described below may also be composed of identification factors that indicate locations. Namely, the single-gene haplo frequency information 412 provides the frequency of each gene in 39,000 human genes and 5000 people of the world races and annotation information on a variety of diseases.

Furthermore, the multiple-gene information database 420 is a database configured to store variation distribution and information on multiple genes, and comprises a multiple-gene haplo map 424 and multiple-gene haplo frequency information 422.

In this regard, the multiple-gene haplo map 424 stores variation distributions classified by proportion, for related nucleotides of the entire control group for each of phenotypes specified by multiple genes, and summarizes the results of calculating the haplotypes of 26 world races by use of phenotype-causing variants and calculating the frequency of a specific trait and the frequency of each sub-race.

Furthermore, the multiple-gene haplo frequency information 422 stores information on each variation. In this regard, the multiple-gene haplo frequency information 422 can also directly store variation information, and information stored in the information DB 800 to be described below may also be composed of identification factors that indicate locations.

Namely, the multiple-gene haplo frequency information 422 provides the frequency of phenotype-related gene sets in 3,9000 human genes and 5,000 peoples of the world races and annotation information on a variety of diseases.

Referring to the example shown in FIG. 6, the X-axis of the haplotype DB 400 represents 3 billion nucleotide sequences, and there are 39,000 nucleotides in then nucleotide sequence. When N variations are found in a specific gene (i) in the schema thereof, the variations can be clustered using all the haplotypes and genotypes of 5,000 people (Y-axis). The clustered form becomes HaploMap.

In this regard, each class means each genotype. Regarding this, the first GP*47*0 means the genotype accounts for 47% of the world population and is 0-bit different from the world population' average (that is, equal). The second GP*25*1 indicates that the genotype accounts for 25% of the world population and is 1-bit differ from the world population' average.

In addition, the multiple genome-based HaploMap is also classified in the same manner.

As shown in FIG. 4, the allele depth DB 500 is a DB that stores genome information on the control population. Specifically, as the population genome, genome information known by performing the global genome project may be used.

Meanwhile, the allele depth DB 500 stores information on the whole genome of the control population, and the information can be classified by criteria forming a group of genotypes, such as race, and can be stored in the allele depth DB 500.

In this regard, the classification by race may be classification into 5 major classes, or classification into 26 subclasses. This is to determine/detect the presence of mutation gene by reflecting the genetic traits of each race.

In addition, as shown in FIG. 4, the IDA DB 600 stores already known diseases and genetic variations related thereto. Specifically, for various diseases, information on genetic variations related to each disease and document information supporting the variant information can be summarized and stored in the IDA DB 600.

Furthermore, the BAV/biomarker DB 700 may store genetic information that determines the types of amino acids at binding positions of various proteins.

Specifically, it stores information on amino acids influencing protein-drug binding, protein-DNA binding and protein-protein binding and information on genes influencing these amino acids.

Accordingly, when a large number of variations in nucleotides in amino acids responsible for the binding of a specific metabolite occur, normal in vivo treatment of the corresponding metabolite in the subject from which the analysis date were obtained will be highly difficult.

The BAV/biomarker DB 700 stores information on physiological activity-related genes. Specifically, information on genes and on resistance and sensitivity to drugs, metabolites and foods is stored therein. In this regard, the BAV/biomarker DB 700 may be constructed by linking data known to be reliable. For example, it may be constructed using information on about 6,000 drugs known in drug banks (information on interacting proteins and binging regions, etc.), information on about 12,000 metabolites known in metabolite banks (information on interacting proteins and binging regions, etc.), and information on the drug metabolism-related variation positions) of about 200 genes present in DMET (drug metabolizing enzyme and transporter gene).

Meanwhile, the information DB 800 is a DB that stores information on known genomic variations, and can be constructed in association with published information database as well as document information.

For example, PheWAS-GWAS (genome wide association study) data and eMERGE (Electronic Medical Records and Genomics) data may be applied to the information DB.

Meanwhile, although not shown, the search control unit 200 may further comprise a clinical information DB that stores subject's environmental factor information to be considered together with genetic traits in order to produce the results of disease cause prediction based on clinical information.

In this case, the clinical information DB stores the result data of personal environmental factors and the population mean and baseline information.

In addition, the result data of personal environmental factors may be clinical information data such as personal comprehensive medical examination data, and the population average and baseline information may be based on the results of community cohort studies provided by the Centers for Disease Control and Prevention.

Hereinafter, the method of analyzing genetic information by use of a personal genome according to the present invention will be described in detail with reference to the accompanying drawings.

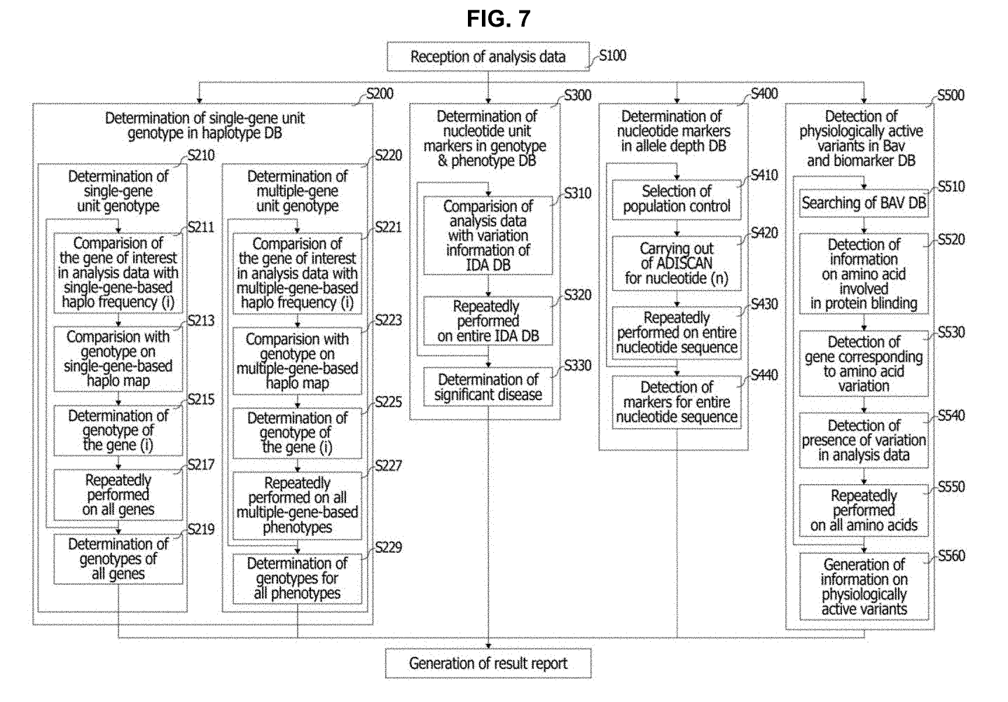

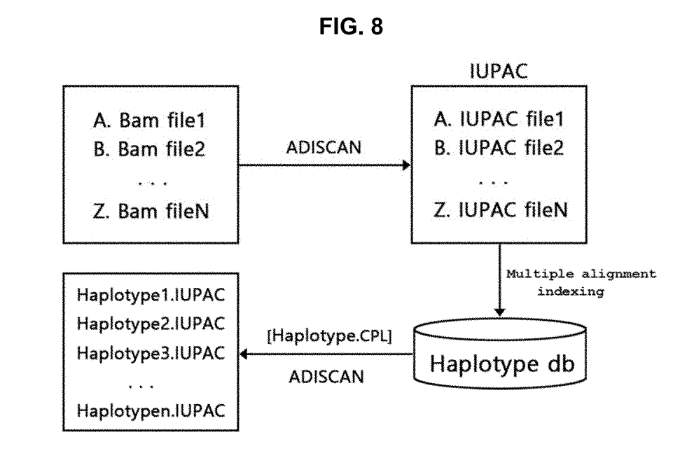

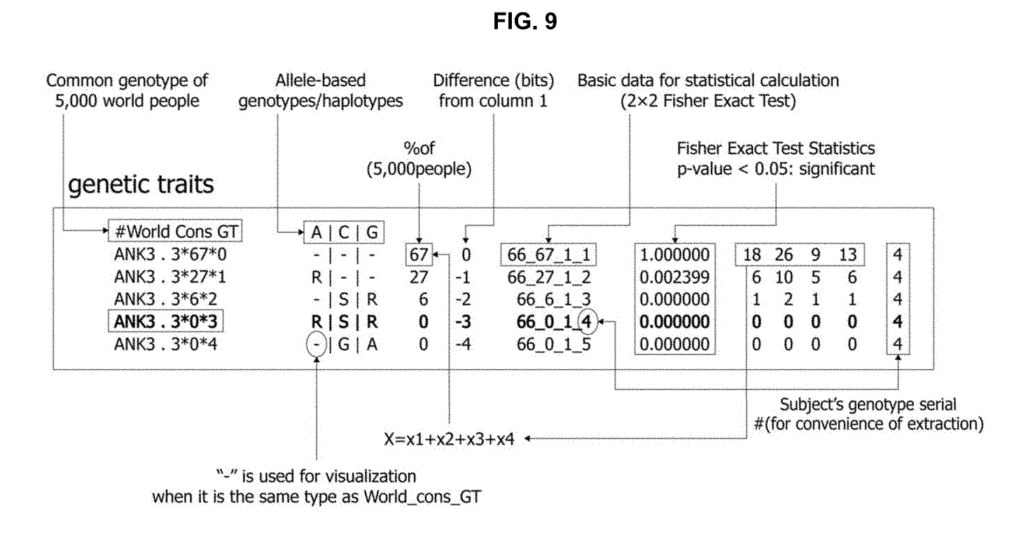

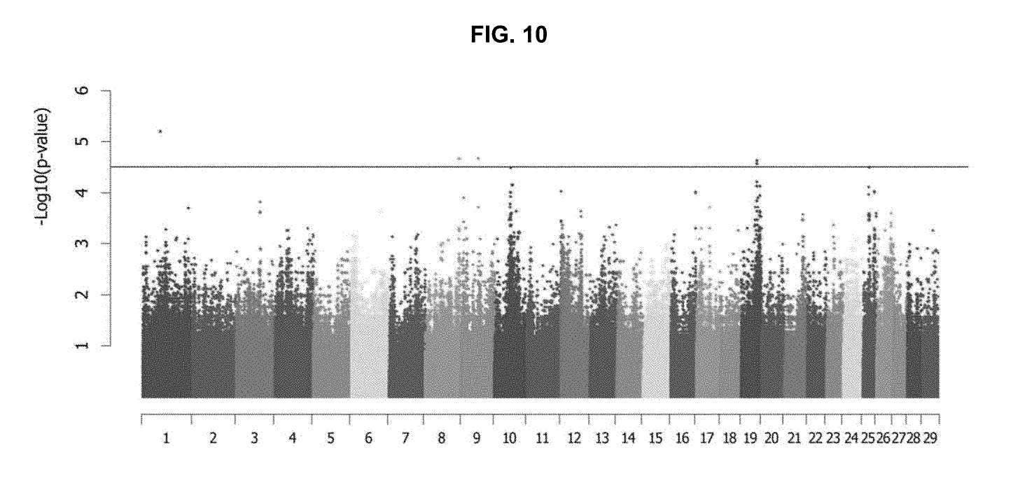

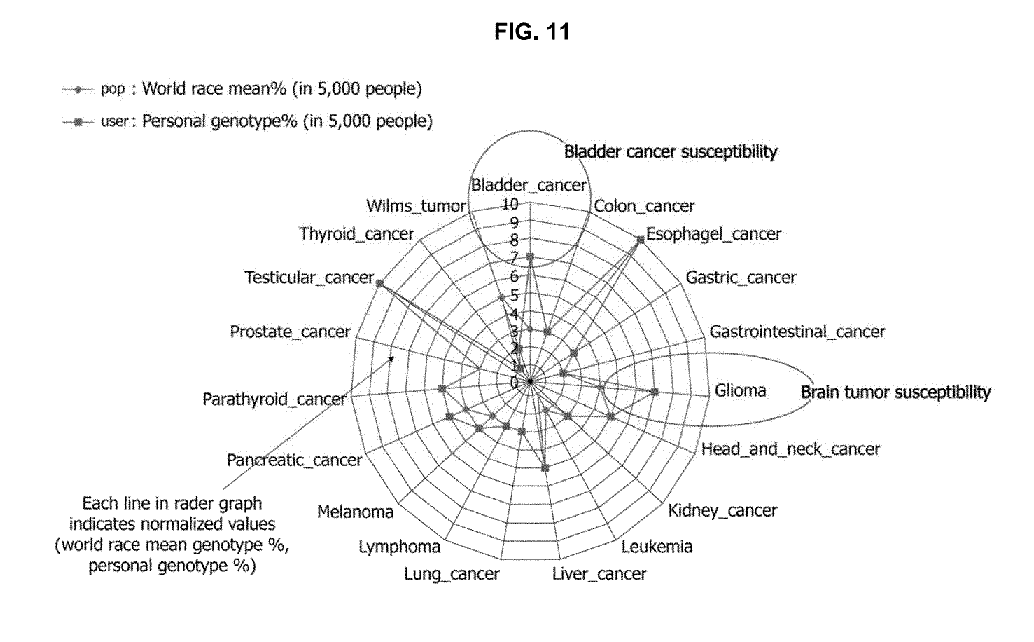

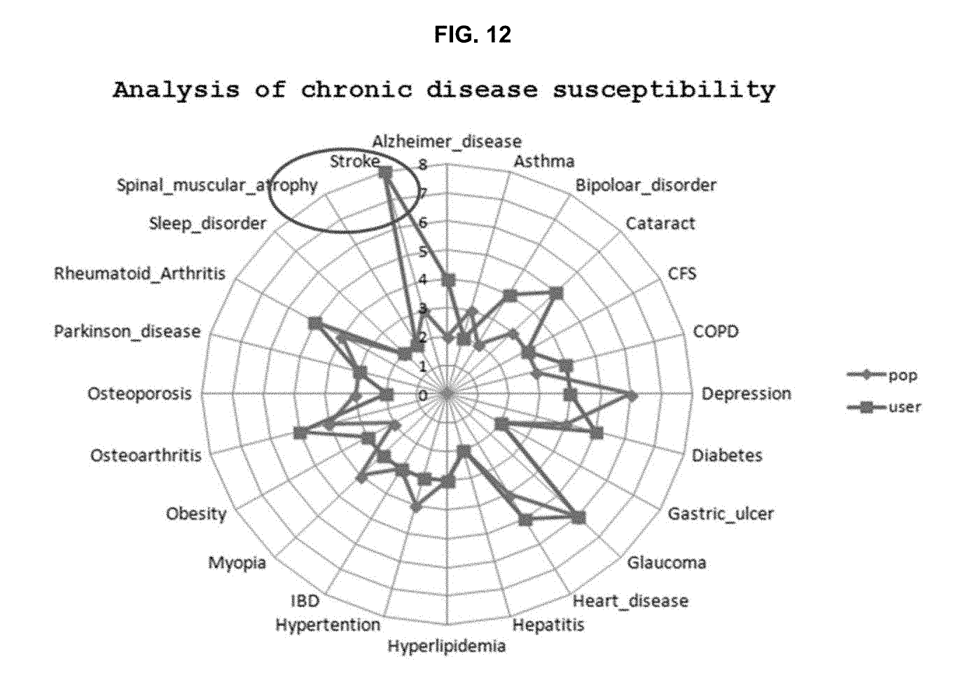

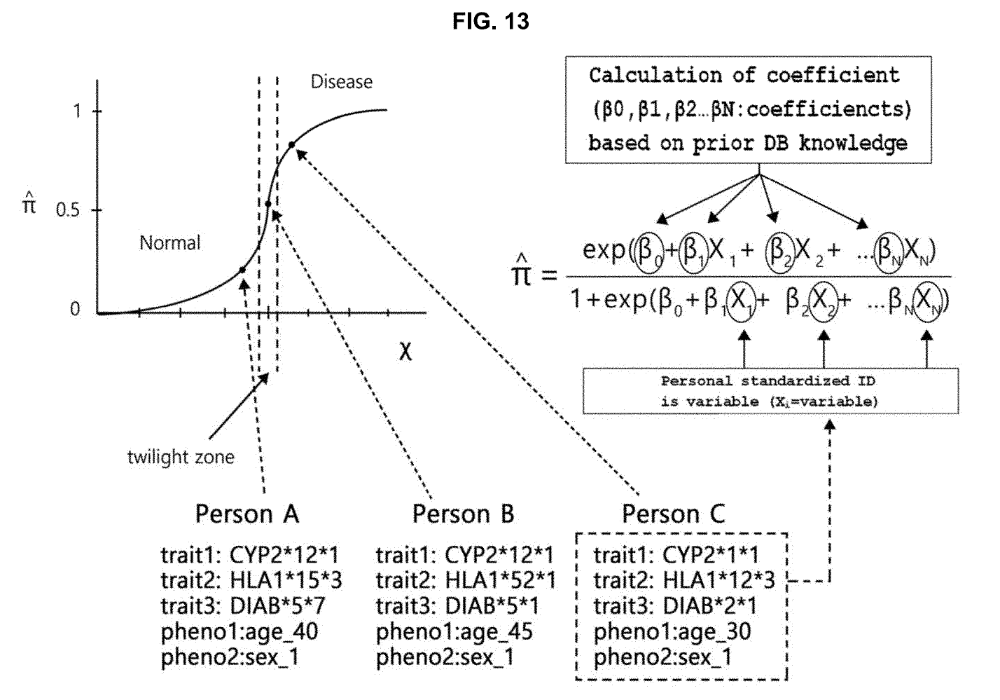

FIG. 7 is a flow chart showing a method for genotype analysis according to a specific embodiment of the present invention; FIG. 8 illustrates an example of the generation of a haplotype DB according to a specific embodiment of the present invention; FIG. 9 illustrates an example of genotype analysis results produced according to a specific embodiment of the present invention; FIG. 10 illustrates an example of a Manhattan plot of a result report produced according to a specific embodiment of the present invention; FIG. 11 illustrates an example of a radar mutation significance chart of a result report produced according to a specific embodiment of the present invention; FIG. 12 illustrates another example of a radar mutation significance chart of a result report produced according to a specific embodiment of the present invention; and FIG. 13 is a conceptual view showing a system for computing the cause of disease and drug (food) response based on clinical information according to a specific embodiment of the present invention.

As shown in FIG. 7, in the method for genotype analysis by use of information on genetic variation of a personal genome according to the present invention starts with a step in which the analysis data input unit receives analysis data (DNA sequencing data) (S100).

In this regard, the analysis data may be provided as a dummy composed of DNA fragments. In this case, as shown in FIG. 8, DNA sequencing is produced in the RVR format through highly integrated indexing and stored in the provided dummy data.

FIG. 8 shows an example of the generation of a haplotype DB. Specifically, it shows an example of extracting population genetic information and parameters at corresponding positions from the haplotype DB.

Specifically, using the genomic information, genotype files in the IUPAC format are generated from a binary alignment map (BAM) file through ADISCAN. In addition, an indexed database of indexed multiple nucleotide alignments is constructed, and then IUPAC information, population genetic information and parameters at corresponding positions are extracted from the haplotype DB by use of a chromosome position list (CPL).

Next, the method for genetic information analysis according to the present invention analyzes the genotype of the analysis data.

In this regard, the analysis of the genotype comprises analyzing the genotype of each gene of the personal genome in the analysis data and analyzing the genotype of a combination of multiple genes that appear as phenotypes.

[Determination of Genotypes of Single Genes]

Determination of the genotypes of single genetic units comprises calculating the ID of haplotypes of genetic units (LD block, exon unit, gene marker, etc.) in the haplotype DB, and the HaploScan engine 210 compares the haplo frequency 412 of the i^{th} gene in the DNA sequencing with that of the i^{th} single gene stored in the haplotype DB 400 (S211).

Then, variation information on the i^{st} gene in the DNA sequencing is acquired, and it is determined where the i^{th} gene is contained in any of the single-gene classes included in the single-gene Haplo MAP 414 (S213, S215). Thereafter, the HaploScan engine 210 repeats the above procedure from i=1 to the last (about i=39,000), thereby determining the genotype of the entire genes of the analysis data (S217, S219).

[Determination of Genotypes of Multiple Genes]

Determination of the genotypes of multiple genetic units comprises calculating the ID of haplotypes of multiple genetic units (multiple gene markers, GWAS markers) in the haplotype DB, and the HaploScan engine 210 compares the DNA sequencing with the haplo frequency 422 of the multiple genes (S221).

Then, it is determined where a combination of the multiple genes of the genome to be analyzed for the corresponding phenotypes is contained in any of multiple-gene combination classes included in the multiple-gene haplo MAP 424 (S223, S225).

Thereafter, the HaploScan engine 210 repeatedly performs steps 221 to 225 on all the phenotypes stored in the multiple-gene information database 420, thereby determining the genotype of the multiple-gene combination in the analysis data (S227, S229).

Through the HaploScaning process as described above, the genotype resulting from the single-gene variation and multiple-gene variation included in the genome to be analyzed can be defined.

FIG. 9 shows an example of the results of determining the genotype of the analysis through the above-described process. As shown therein, the determination results include a class to which the corresponding genotype pertains, allele-based haplotypes of the corresponding class, the level of significance, and the like.

Namely, as shown in FIG. 9, in the results of genetic variation of personal genome detected by the HaploScaning process, the location of the genotype (ANH, 3*0*3) to be analyzed corresponds to the fourth line, and the statistical significance (p-value) of the fourth line is less than 0.05. Thus, the genotype to be analyzed can be interpreted as having significance.

In addition, when known genetic traits (e.g., disease-related variation) are found in the variations to be analyzed, it can be determined that the genetic traits have susceptibility.

R in R|*S|*R is known as a cancer-susceptibility disease variation, and is an example of calculating a genetic variation with disease susceptibility by the analysis system of the present invention.

Meanwhile, the search control unit 200 can generate a result report based on the determined genotype of the analysis data.

The result report generally uses a Manhattan plot and a radar chart visualize variant genes, even though there is a somewhat difference depending on products.

FIG. 10 illustrates an example of a Manhattan plot generated according to a specific embodiment of the present invention.

As shown in FIG. 10, the Manhattan plot refers to a graph obtained by classifying the standard genes of the genome project by genotype on the basis of all known SNP non-sym variations for 39,000 genes and expressing cumulative values as points.

When the genomic gene to be analyzed is expressed therein, the variation specificity of the gene to be analyzed compared to the control can be easily recognized.

The use of this Manhattan plot makes it possible to easily recognize not only variation loci but also the level of variation.

Meanwhile, significant variations indicated by the Manhattan plot may be expressed as a radar variation chart depending on the level of the variation and genetic traits as shown in FIGS. 11 and 12.

In this case, the variation level of the genome to be analyzed is indicated together with the control mean, and thus the variation level of the genome to be analyzed can be visibly and clearly expressed, and a result report further comprising genetic traits can also be generated.

The result report produced by the above-described method is provided through a result report provision unit.

Meanwhile, the search control unit 200 can determine and provide clinical information-based disease causes based on subject's clinical information, if provided.

Specifically, predicting the cause of disease requires PHR (personal health records) that includes current environmental factor consequences (comprehensive medical examination data and clinical information). Particularly, the population mean and baseline information in environmental factors is required (in the present invention, stage-2 community cohort study results provided by the Centers for Disease Control and Prevention). Here, an association of these environmental factor results with genetic traits is called PHR-trait.

As shown in FIG. 13, the disease cause relationship (Π) is determined by logistic regression analysis. Herein, variable β is a value determined by the genetic traits calculated as described above, and variable χ is a value determined from the PHR.

Namely, the disease cause relationship makes it possible to calculate the correlation of gene, disease or drug with genotypes (a group or cluster of genotypes vs. PHR (BMI, AGE, SEX, etc.).

Thus, a disease cause based on entire genes is calculated by calculating the correlation between current clinical conditions (normal, disease, or phenotype) and gene, disease or drug genotypes calculated for 39,000 genes.

Namely, as shown in FIG. 13, the disease cause relationship (Π) is determined by logistic regression analysis, and the arithmetic expression for disease cause relationship (Πx) is

$${π}_{x} = \frac{exp ( {β}_{0} + {β}_{1} {x}_{1} + {β}_{2} {x}_{2} + … + {β}_{n} {x}_{n} )}{1 + exp ( {β}_{0} + {β}_{1} {x}_{1} + {β}_{2} {x}_{2} + … + {β}_{n} {x}_{n} )} .$$

Genotypes or personal profiles (standardized ID set) are calculated using a variety of given ID generation systems through population genomes and their EMR (electronic medical record), HER (electrical health record) and PHR (personal health record), and coefficient variable β is generated using a given ID system. Furthermore, personal information generates personal profiles (standardized ID set) by using the personal genome and the hospital-based phenotype information on the person as standards, and the IDs provide variable χ to the arithmetic expression determined by multiple logistic regression.

Namely, the disease cause relationship makes it possible to calculate the correlation of gene, disease or drug with genotypes (a group or cluster of genotypes vs. BMI, AGE or PHR).

Thus, a disease cause based on entire genes is calculated by calculating the correlation between current clinical conditions (normal, disease, or phenotype) and gene, disease or drug genotypes calculated for 39,000 genes.

Meanwhile, the method for genotype analysis using genetic variation information on a personal genome according to the present invention may comprise: (S300) detecting nucleotide unit markers in the IDA DB; (S400) detecting nucleotide unit markers in the allele depth DB; and (S500) calculating physiologically active variants.

[Detection of Nucleotide Unit Marker in IDA DB]

Detection of nucleotide unit makers in the IDA DB comprises calculating disease and drug response by use of the genotype and phenotype information and detecting significant information. For detection of nucleotide unit markers in the IDA DB, the IDA search engine 230 compares the analysis data with the variation information included in the IDA DB 600, thereby determining the risk of the corresponding disease (S310).

According to this method, the analysis data are reviewed for all diseases included in the IDA DB (S320), and significant variation-related diseases are detected (S330).

[Detection of Marker Unit Markers in Allele Depth DB]

A nucleotide unit marker is a nucleotide variation caused by an extremely unusual specific genetic variation, and is often related to rare diseases. Detection of nucleotide unit markers in the allele depth DB makes it detect the presence or absence of a variation in a specific base and determine the possibility of developing a rare disease.

To this end, as shown in FIG. 7 according to the present invention, the ADISCAN engine 220 first selects a control group (S410).

Here, the control group is a control group to be used to determine the rarity of a corresponding variation, and may also be limited to a particular race or a specific nation.

Next, the ADISCAN engine 200 produces a variation index for the nucleotide at a specific locus by use of the nucleotides of the control DB and the ADISCAN method, and this process is performed for the whole genome (from n=1 to n=about 30 billion) (S420, S430).

Accordingly, the rarity of nucleotides for the entire nucleotide sequence is determined (S440).

Meanwhile, the ADISCAN (allelic depth and imbalance scanning) for determination of rare variations is a technique of screening markers that are different between normal and abnormal genes. Here, the determination is performed based on allele depth multiply tangent difference, allele squared difference, allele absolute value difference, geometric allele difference, statistical allele difference or allelic imbalance ratio.

[Detection of Physiologically Active Variants]

Detection of physiologically active variants comprises calculating the significance of various markers compared with the BAV/biomarker DB and common markers. To this end, the physiological activity variant search engine 240 searches the BAV/biomarker DB (physiological activity variant DB) (S510) and detects information on amino acids involved in protein binding (S520).

In this regard, the protein binding include protein-drug binding, protein-DNA binding and protein-protein binding, and the information on amino acids includes information on nucleotides related to the amino acids.

Then, the physiologically active variant search engine 240 detects compares nucleotides included in the amino acid information with the analysis date, thereby detecting information on the amino acids in which variation has occurred on the analysis data and metabolites related thereto (S530, S540).

Furthermore, the physiologically active variant search engine 240 repeatedly performs variation detection on all the amino acids, and integrates the detected information, thereby generating information on physiologically active variants (S550, S560).

The scope of the present invention is not limited to the above-described embodiments, but is defined by the appended claims, and those skilled in the art will appreciate that various modifications and alterations are possible without departing from the scope of the present invention as defined in the appended claims.

The present invention s relates to a system of analyzing and providing genetic information by comparing input personal genomic information with a plurality of whole-genome DBs constructed by genome projects. According to the present invention, a gene analysis platform can be provided which compares genome variations with improved efficiency by applying a database schema including a haplo skin map to a control database.

1-13. (canceled)

14. A system for genotype analysis using genetic variation information on a personal genome, the system comprising:
an analysis date input unit configured to receive analysis data including personal genomic information;
a search control unit configured to produce analysis results including a genotype of each gene or genotype versus phenotype by comparing genetic information stored in a database with the analysis data and to generate a result report based on the analysis results; and
a storage unit comprising a HaploScan DB that stores genotype information on genes of a control group to compare with the analysis data,
wherein:
the search control unit comprises a HaploScan engine configured to determine the genotype of the analysis date by comparing the analysis data with the haploScan DB;
the HaploScan DB comprises:
a single-gene information database that stores genotype information on single genes; and
a multiple-gene information database that stores genotype information on multiple genes for each genotype;
the single-gene information database comprises:
a single-map haplo map that stores haplotype and trait frequencies for each race, classified (clustered) by proportion, for single genes of the control group; and
single-gene haplo frequency information that stores variation information on variations that classify the single-gene genotypes stored in the single-gene haplo map;
the multiple-gene information database comprises:
a multiple-gene haplo map that stores genotype-associated nucleotide variation distributions classified by race and proportion for multiple genes of the control group for each phenotype; and
multiple-gene haplo frequency information that stores variation information on variations that classify genotypes for the phenotypes stored in the multiple-gene haplo map;
the storage unit further comprises a clinical information DB that stores subject's environmental factor information to be considered together with genetic traits in order to produce the results of disease cause prediction based on clinical information;
the search control unit is configured to produce the results of disease cause prediction by generating a disease cause relationship (Πx) through an arithmetic expression generated by logistic regression;
the arithmetic expression for the disease cause relationship is
$${π}_{x} = \frac{exp ( {β}_{0} + {β}_{1} {x}_{1} + {β}_{2} {x}_{2} + … + {β}_{n} {x}_{n} )}{1 + exp ( {β}_{0} + {β}_{1} {x}_{1} + {β}_{2} {x}_{2} + … + {β}_{n} {x}_{n} )}$$
wherein
variables β are parameters dependent on subject's personal health records (PHRs), including age, sex or bone mass index, stored in a clinical information DB; and
variables χ are parameters dependent on either the genotypes of single genes included in the analysis data produced by the search control unit or the genotypes of multiple genes for each phenotype.

15. The system of claim 14, wherein the result report comprises an index indicating the level of significance compared with the classified region (class) to which the genotype of the analysis data belongs.

16. A method for genotype analysis using genetic variation information on a personal genome, the method comprising:
step (A) in which an analysis date input unit receives analysis data consisting of DNA sequencing data;
step (B) in which a HaploScan engine determines genotype of a gene included in the analysis data;
step (C) in which the HaploScan engine acquires variation information on the gene of the analysis data;
step (D) in which step (B) and step (C) are repeatedly performed on all genes included in the analysis data; and
step (E) in which the search control unit produces the results of disease cause prediction by generating a disease cause relationship (Πx) through an arithmetic expression generated by logistic regression;
wherein:
the determination of the genotype in step (B) comprises:
a step of determining the genotype among genotype classes classified in a single-gene haplo map, for single genes of the analysis data; and
a step of determining the genotype among genotype classes classified in a multiple-gene haplo map, for multiple genes included in the analysis data;
the acquisition of the variation information in step (C) comprises:
a step of comparing single-gene haplo frequency information on a gene at a specific locus in the analysis data with that on a gene at the same locus, thereby acquiring variation information on the gene at the specific locus in the analysis data; and
a step of comparing multiple-gene haplo frequency information on multiple genes of the analysis data with that on multiple genes for a specific phenotype, thereby acquiring variation information on the multiple genes of the analysis data;
the single-gene haplo map stores haplotype and trait frequencies for each race, classified (clustered) by proportion, for single genes of the control group;
the multiple-gene haplo frequency information stores variation information on variations that classify the single-gene genotypes stored in the single-gene haplo map;
the multiple-gene haplo map stores multiple-gene variation distributions of the control group for each phenotype, classified by proportion;
the multiple-gene haplo frequency information is variation information on variations that classify genotypes for the phenotypes;
the arithmetic expression for the disease cause relationship is
$${π}_{x} = \frac{exp ( {β}_{0} + {β}_{1} {x}_{1} + {β}_{2} {x}_{2} + … + {β}_{n} {x}_{n} )}{1 + exp ( {β}_{0} + {β}_{1} {x}_{1} + {β}_{2} {x}_{2} + … + {β}_{n} {x}_{n} )}$$
wherein
variables β are parameters dependent on subject's personal health records (PHRs), including age, sex or bone mass index, stored in a clinical information DB; and
variables χ are parameters dependent on either the genotypes of single genes included in the analysis data produced by the search control unit or the genotypes of multiple genes for each phenotype.

17. The method of claim 16, further comprising step (F) in which the search control unit generates a result report based on the produced results.

18. The method of claim 17, wherein the result report comprises an index indicating the level of significance compared with the classified region (class) to which the genotype of the analysis date belongs.
